(19) United States
(12) Patent Application Publication
(10) Pub. No.: US 2016/0367015 A1
(43) Pub. Date: Dec. 22, 2016

(54) DEVICE TO OPTIMIZE COSMETIC INJECTABLE PROCEDURES

(71) Applicant: AESTHETIC MEDICAL DEVICES, LLC, Sherman Oaks, CA (US)

(72) Inventors: GEORGE HUNG, SHERMAN OAKS, CA (US); WAYNE CHUNG, PITTSBURGH, PA (US); DAVID SAWCER, PASADENA, CA (US)

(73) Assignee: AESTHETIC MEDICAL DEVICES, SHERMAN OAKS, CA (US)

(21) Appl. No.: 14/900,987

(22) PCT Filed: Jul. 2, 2014
(86) PCT No.: PCT/US2014/045313

§ 371 Date: Dec. 22, 2015

Related U.S. Application Data
(60) Provisional application No. 61/842,812, filed on Jul. 3, 2013.

(51) Int. Cl. A45D 44/22; A61B 90/00; A61F 2/00

(52) U.S. Cl. CPC A45D 44/22; A61F 2/0059; A61B 90/08; A61B 2090/0817

(57) ABSTRACT

The present invention provides a system consisting of a handheld device with integrated power source. The system will be designed to perform several treatment (massage) modalities and will be used to optimize outcome following cosmetic injectable procedures. The cosmetic injectable procedures that the invention will enhance include but are not limited to: Injection procedures with Sculptra, Radiesse, Restylane and Juvederm and autologous fat transfer injection procedures—as a group these injection procedures are referred to as “volumizing” procedures. The practitioner may use this device at the time of the injection procedure and the patient may use the system for a defined period of time thereafter—the timing and duration of use will depend on the procedure performed and the professional consideration of the practitioner performing it. The use of the device system may also include various serums and lotions that will enhance treatment with the device.

### FIELD OF THE INVENTION

Embodiments of the present invention relate to portable lightweight handheld integumentary treatment devices which in this case both in design form and design materials substantially mirror the hardness and deformation characteristics of the human thumb or index finger. It is intended that this device be uniquely effective in, contouring, sculpting and finessing cosmetic injectable materials to enhance aesthetic outcomes and also to prevent and treat lumpiness and other similar adverse events known to be potentially caused by the injectable materials used in volumizing procedures.

### BACKGROUND OF THE INVENTION

The art of massage, the manipulation of superficial and deeper layers of the body, uses various techniques to enhance function, aid in the healing process, decrease muscle reflex activity, inhibit motor-neuron excitability, promote relaxation and increase well-being. A number of professional practitioners, in a variety of medical settings, provide therapeutic massage including, athletic trainers, chiropractors, physical therapists and practitioners of many traditional Chinese and other eastern medicines. A number of devices are used to enhance several massage techniques.

Over the past 20 years, volumizing injectable procedures have become part of the standard of care for treating significant volume loss both reconstructively (following injury or illness) and cosmetically (addressing and correcting the natural volume loss that occurs with aging). A number of injectable materials are used for these procedures including hyaluronic acid, poly-L-lactic acid, calcium hydroxyl apaitate collagen and autologous fat. Some of the most frequently used products containing these materials include Restylane and Juvederm Sculptra and Radiesse. Materials are injected into the patient by trained physicians. Injections may be given at various depths within the skin and superficial subcutaneous tissues, at sites of significant appreciable volume loss and where subsequent volume correction is judged likely to be beneficial. The effect, depending on the product material, is to add immediate volume or induce a subsequent volumizing reaction within the skin that later has the same desired volumizing effect thereby in either case correcting the assessed deficit.

An immediate (at the time of the procedure) adverse event associated with injectable volumizing procedures is formation of an unwanted “lumps” or “bumps” and or a “textural unevenness” caused by a greater than desirable deposition or clumping of the injected material at a given site. This unwanted effect can be felt with gentle pressure on the skin by the physician (and consumer) and may even be noticeable visually in more extreme cases. Such adverse events may occur in spite of careful injection technique and when noted are managed by local directed massage performed by the physician at the time of the injection procedure and or by the patient for a defined period of time thereafter—the timing and duration of such massage will depend on the procedure performed and the professional consideration of the practitioner performing it. Lumps, bumps and unevenness may also occur as a later stage phenomena appearing several weeks to months following an injection procedure. This occurs most notably with longer lasting products such as Radiesse (calcium hydroxyl apaitate) and Sculptra (poly-L-lactic acid). A number of efforts are effective to minimize this later stage phenomena when used at or close to the time of the procedure; including careful injection technique, reconstitution or dilution in a larger volume and in the case of Sculptra allowing the material reconstitute over a long period of time. Notably many physicians recommend that the patient massage the injected site several times per-day for several days after the procedure to minimize lump formation later.

Documented incidence of lump and bump formation for several products from their current (Accessed June 2013) FDA prescribing information is shown below:

| | | | Lumps and Bumps |
|---|---|---|---|
| | Product | Company | Incidence |
| Hyaluronic Acid | Juvederm | Allergan | 79%; 75% |
| | Restylane | Valeant/ | 90% |
| | | Medicis | |
| | Prevelle Silk/ | Mentor | 50% |
| | Puragen | | |
| Poly-L-Lactic Acid | Sculptra | Valeant | 17.20% |
| Calcium Hydroxyl | Radiesse | Merz | 18% |
| Apaitate | | | |
| Collagen | Artefill | Suneva | 4.60% |
| | | Medical | |
| | Zyderm/Zyplast | Allergan | 20% |

Procedures producing an immediate volumizing effect such as those using hyaluronic acid (Restylane and Juvederm) and collagen, almost always need a degree of massage to produce contouring and sculpting, finessing the injected material to achieve the desired aesthetic outcome. The physician will do this at the time of the injection procedure and may direct the patient to do the same in a limited manner for a defined period of time thereafter —again the timing and duration of such massage will depend on the procedure performed and the professional consideration of the practitioner performing it. When using materials that produce an immediate volumizing effect, one of the challenges of massaging by either the physician or patient is to avoid over-massage of the treatment area, by massaging to vigorously or for too long a period of time. With “over-massage” of the treated area the injected material is displaced into the surrounding tissue so that there remains a barely perceptible improvement in surface contour.

There are no currently marketed portable lightweight handheld integumentary treatment devices known in the art that have been specifically designed with unique properties to treat immediate unwanted lumps, bumps or textural unevenness, that assist in contouring, sculpting and finessing injected material to achieve the desired aesthetic outcome, or that minimize later lump formation, while at the same time helping the physician and patient to avoid over-massage of the treatment area.

### SUMMARY OF THE INVENTION

The present invention is generally directed to a massage device which substantially mirrors the hardness and deformation characteristics of the human thumb or index finger in order to manipulate injectable materials and be particularly effective in treating immediate unwanted lumps, bumps or textural unevenness, in contouring, sculpting and finessing injected material to achieve a desired aesthetic outcome, in preventing or minimizing later lump formation, and at the same time helping the physician and patient to avoid over-massage of the treatment area. The device system typically includes a portable lightweight handheld integumentary treatment device that can fit comfortably in a user's hand. The device will include several surfaces of varying curvature, surface area and composition that can be used in conjunction with various massage techniques. It will include an on/off button and design elements to prevent the use of excessive force and or use for an over-extended period of time.

The device system may use various modalities or combination of modalities to help improve the outcomes of procedures including: vibration energy, heat energy, electrical energy, optical illumination energy, infra-red energy, and ultrasonic energy. The delivered energy acts via physical and/or physiological mechanisms to optimize treatments, improve the outcome and reduce the frequency of adverse events

The device system will be specifically designed to optimize (compliment) several massage techniques. The design will include multi-surface contact areas of varying contour and optionally of variable texture and variable softness/rigidity that range from broad and sweeping to more angled and acute. It will include a larger gently curved surface area connected to a smaller more acutely curved surface area that meet at an angled curving tip that will all serve as contact areas. The purpose of these multi-surface (varied-contour) contact areas is that they will be used for different massage techniques and allow optimal positioning of the device during contact that matches the curvature of the anatomical surface being treated.

The device system will be carefully designed to include structural and material elements to reduce the risk of over-massaging the treatment area thereby reducing the intended “volumizing” benefits of the injection procedures. By careful structural design and material(s) selection the device will reduce any excessive force that the device user might unintentionally deliver to the volumizer material while still ensuring transmission the various types of energy being applied. The structural design may include a section of the device that contains a hinge or flexible member which interconnects that part of the device touching the skin surface and the portion of the device held in the hand. This intermediate section will be designed in a manner so as to offset any excessive force exerted by the user. In addition to use of a hinge or flexible member another there will be careful selection of the material(s) forming the region of the device that contacts the skin.

Key properties that will be considered when selecting the material(s) from which the devices contact surfaces are manufactured will be the malleability of the material and the amount of friction the material(s) will generate when in contact with the surface of the skin. The material(s) properties will be chosen to maximize comfort, optimize the treatment and again contribute toward offsetting any excessive force exerted by the user.

An additional option to prevent over-treatment is embedding a sensor, which alone or together with a processing circuit can indicate the user when an excessive force is applied, or when the device is applied for excessive period of time.

### BRIEF DESCRIPTION OF THE DRAWINGS

The invention will be better understood, by way of example, with reference to the accompanying drawings, in which:

FIG. 1a is a schematic view of the device;

FIG. 1b is a perspective view of an illustrative embodiment of the massage device of the present invention;

FIG. 1c includes various curvatures of the device and respective size and position of the thumb and index finger for control of the device;

FIG. 2a is one potential design of the device;

FIG. 2b is a design that has a bump that could house elements that could mitigate use of too much pressure;

FIG. 2c is a third potential design of the device;

FIG. 3a is a preliminary sketch of the device;

FIG. 3b is another preliminary sketch of the device;

FIG. 3c is a sketch of the device that shows where the design elements can be housed to mitigate overuse of force;

FIG. 3d is another sketch of the device that shows where the design elements can be housed to mitigate overuse of force.

### DETAILED DESCRIPTION OF THE INVENTION

The above described drawing and figures illustrate the invention in at least one of its preferred embodiments, which is further defined in detail in the following description.

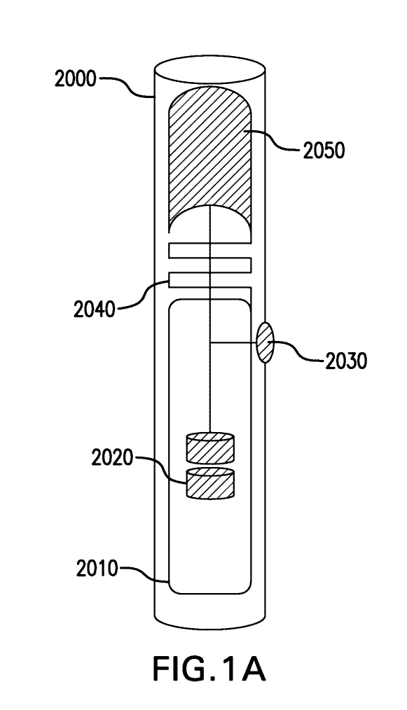

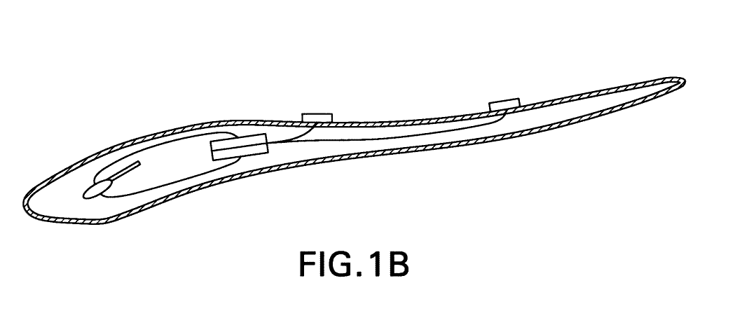

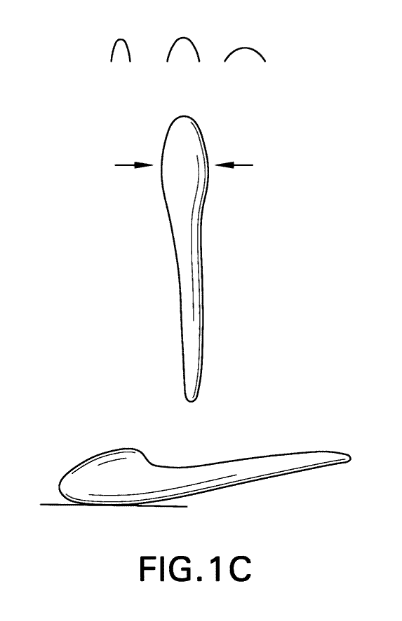

Referring initially to FIG. 1a of the drawings, the device includes a generally elongated body (2000), a “handle” section of the device (2010), batteries (2020), an on/off button (2030), a connector or neck (2040) that links the handle to the head of the device, the “head” of the device (2050). The device will be designed and constructed such that most commonly when used the surfaces of the “head” (2050) are the sites placed contact with the site being massaged and the “handle” (2010) is the site held in the user's hand—but the two portions of the device are intended to be somewhat interchangeable during regular use.

Body cavity (2000) is preferably formed as a plurality of molded plastic pieces, the pieces coupled together in a manner which firmly seals the body cavity. Sealing the various components of motion assembly (2050) and power assembly (2020) within body cavity is contemplated to increase durability and therefore life of motion assembly (2050) and power assembly (2020) by preventing dirt, water, or other substances from affecting these parts and also prevents any chemicals or parts from the components adversely affecting a user of the device.

Head (2050) and handle (2010) comparably have a size and shape similar to a common toothbrush or small hand held face massager. The handle cavity (2010) must be sized and dimensioned to be held comfortably and firmly in the user's hand. It must also be sufficiently sized to securely hold power source (2020). Head cavity must also be sized and shaped to position and permit proper operation of the device to be used with several massage techniques in order to optimize benefits of subdermally injected materials. In one embodiment of the device, the head cavity may contain a simple electric motor that delivers a gentle vibration energy through the head surface of the device to the user's face. It is contemplated that the head of the device may vary between embodiments. The head of the device may include elements for production and delivery of various energy modalities or combination of modalities to optimize treatment outcomes including: vibration, heating, electrical stimulation, optical illumination, infra-red and ultrasonic energy. While as noted previously the device will be designed and constructed such that physically the head (2050) and handle (2010) during normal use are somewhat interchangeable, the features of the design noted here will remain the same in orientation.

Neck (2040) is preferably sized and dimensioned to allow a hinge or flexible member to connect the handle of the device to the head of the device. This hinge or flexible member may by a spring that is designed to reduce the maximal pressure that the user can apply through the “handle” (2010) to the “head” (2050) of the device. The spring or alternate structure may have a sensor imbedded in it to detect forces applied to the device. It is contemplated that the structure of the neck (2040) may vary between embodiments, the variance in structure being to accommodate the various energy modalities and structural types of head design.

In addition to structural designs to detect and respond to various pressure and forces, it is contemplated the neck of the device could include a pressure sensor and include several embodiments to notify the user that excessive force is being used. These signaling methods could be a sound or vibration. There could be a colorimetric signal whereby the material could change color under the index finger in order to train the user when excessive force is being applied. It is contemplated that more complex detection and signaling methods could be used for the device.

Power source (2020) is preferably battery cells such as hearing aid batteries size 675. However, alternate embodiments may use different types of batteries or capacitors as power source (2020). It is preferred that the choice of power source be made such that the amount of time that power source (2020) is able to adequately power the head/motor is at least 6 hours, and preferably at least 8-10 hours. If usage is as high as 25 minutes per day, the operational life of the device is approximately for 2 weeks of use post injection procedure.

Control assembly (2030) may comprise a simple switch used to complete or break an electrical connection (not shown) between power source (2020) and device head/motor (2050). Such connections are well known in hand held flashlights. However, alternate embodiments may use more complex means of motor control.

FIG. 1b illustrates alternate embodiment with a head designed with several contact surfaces of different curvature (shape) and surface area designed specifically for application of different types of massage to the varied curved surfaces of the face (the intended facial sites are those typically injected during volumizing procedures). There is a short surface shown on the top left of the device for one type of massage (for example the surface is optimized for application of varying degrees of pressure in a rolling manner applied directly into the meliolabial fold—an area between the cheek and upper lip beside and below the nose which is the FDA approved site for injection in the case of most volumizing materials). There is a round more pointed surface at the top of the figure for a second set of massage techniques (for example this surface is optimized for application of more localized pressure applied directly onto a lump or bump occurring at any site) and there is a longer surface on the top right of the device (for example a surface is optimized for application of varying degrees of pressure in a sweeping and or rolling manner applying pressure more diffusely over broad surfaces such as the cheeks or temples). While these three surfaces are illustrated the design of the device will be such that a wide variety of different curvatures and surfaces will be available to be used allowing the user to match the surface of the device with the contour of the face and or with the intended purpose.

FIG. 1b also illustrates alternate positions for the on/off switch. It could either be places near the neck of the device and close to the head or lower on the handle and further away from the neck and head of the device.

FIG. 1c shows various tip curvatures for the most pointed surface of the device. This figure also illustrates potential positions for the thumb and index finger in order to improve control of the device. In this embodiment, the device acts as an extension of the index finger and thumb. The area under the index finger could house the neck and pressure sensor that could notify the user that too much force is being exerted.

Referring to FIG. 1c, in typical application the device can be used at the time of an injection procedure and for a short while thereafter to assist in contouring, sculpting and finessing injected material to achieve the desired aesthetic outcome. For example, the user may initially place one of the device surfaces in contact with the injection site—the device surface used being the one that best matches the facial contour at the site of the injected material. With gentle pressure applied to the injection site the user repeatedly rolls the device in a side-to-side motion. It is anticipated that no more than a few seconds of varying degrees of gentle pressure will be required and that the user will be continually adjusting the surface in contact until the desired effect has been achieved. The head of the device is will be applied to the face for about 5-10 seconds (depending on the nature of the injection material and the desired effect). The user will thus be applying continuous but variable pressure with rolling massage, in some embodiments of the device it will be desirable that the surface of the device itself will deform (by 1-2 mm made possible by virtue of the nature of the materials chosen for the surface of the device being of a suitable softness/rigidity). This will allow the device to generally conform better to contour of the face against which the device is pressed. The device will also deflect (by 1-2 cm via the structural elements contained within the neck of the device that may include a hinge or flexible member) to reduce any excessive force that the device user might unintentionally deliver to the volumizer material while still ensuring transmission the various types of energy being applied. Accordingly, the device applies energy and pressure which penetrates the skin and contours the injected material without completely flattening the material so that it is no longer visually evident. As the device thus changes shape, the user continues to roll the device against the face in a generally back-and-forth motion. This procedure is applied as often as is necessary to optimize the procedure. It will be appreciated by those skilled in the art of injecting materials that the device can optimize injection procedures.

Another example of use will be to treat unwanted lumps, bumps or textural unevenness. In this case it is intended the user will likely place the tip of the device in contact with the detected injection site lump with the device held in a manner closer to perpendicular to the skin surface. Gentle but firm pressure will then be applied along the long axis of the device towards the tip and the user will manipulate the device in a side-to-side motion to flatten out the defect. Again it is anticipated that no more than a few seconds of varying degrees of pressure will be required in contact until the desired effect has been achieved. In this case deformation of the device surface (by 1-2 mm made possible by virtue of the nature of the softness/rigidity of the materials chosen for the surface of the device) will be advantageous to maintain evenness in outcome. Any device deflection at the neck will be unwanted and minimized given that the pressure is applied along the device to the tip which is in contact with the skin.

A third example of use will be to prophylactically prevent or minimize delayed formation of lumps, bumps and unevenness that may occur several weeks to months following an injection procedure—most commonly with longer lasting injection materials such as calcium hydroxyl apaitate and poly-L-lactic acid. Amongst the maneuvers noted, many physicians recommend that the patient massage the injected site several times per-day for several days directly after the procedure to minimize such late stage lump formation. Here as was the case in the first example, the user initially places one of the device surfaces in contact with the injection site—the device surface being chosen such that it best matches the facial contour at the site of the injected material—this will typically be one of the longer surface such as that depicted in FIG. 1b on the top right of the device. With gentle pressure the user repeatedly rolls the device in a side-to-side motion as they also sweep the device across the surface of the skin. It is anticipated that this will be continued for several minutes with sustained gentle pressure and during that time the orientation of the device (its long axis) will be varied with the intention of uniformly and generally covering repeatedly with gentle massage the whole of the injection site. (In this case there is no risk of displacing the injected material as was the case either unintentionally in the first example or intentionally in the second example). We anticipate that any deformation (by virtue of the device surface softness/rigidity) may make the experience more pleasurable and additionally, on the broader surfaces typically used in this case, they may be coated to glide more easily over the skin surface in some embodiments. As noted this procedure will be repeated several times per-day for several days after the procedure to minimize such late stage lump formation.

In one embodiment the device may contain a vibrating motor that delivers vibration energy—that will be of particular value when used for the purpose described in the third example of use above to prophylactically prevent or minimize delayed formation of lumps, bumps and unevenness. Other embodiments will include various modalities alone or in combination to help improve the outcomes, these may include: vibration energy, heat energy, electrical energy, optical illumination energy, infra-red energy, and ultrasonic energy. The delivered energy acts via physical and/or physiological mechanisms to optimize treatments, improve the outcome and reduce the frequency of adverse events.

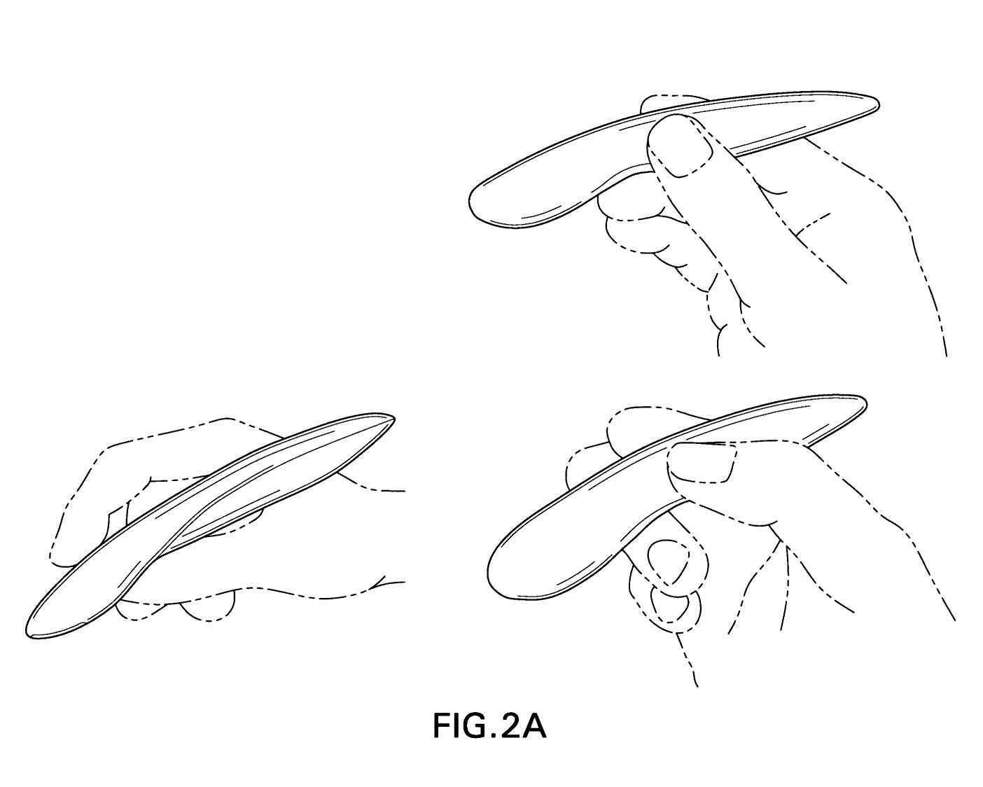

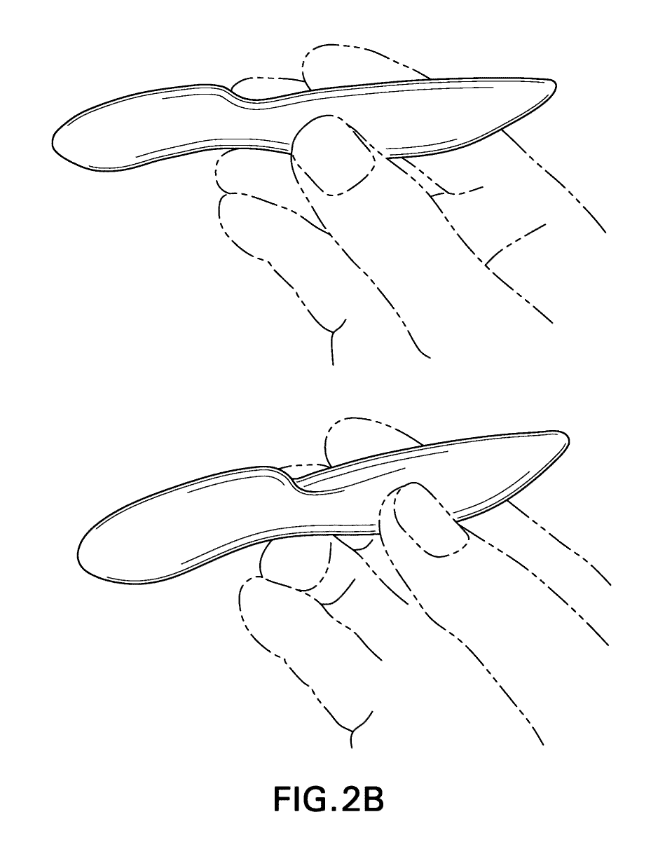

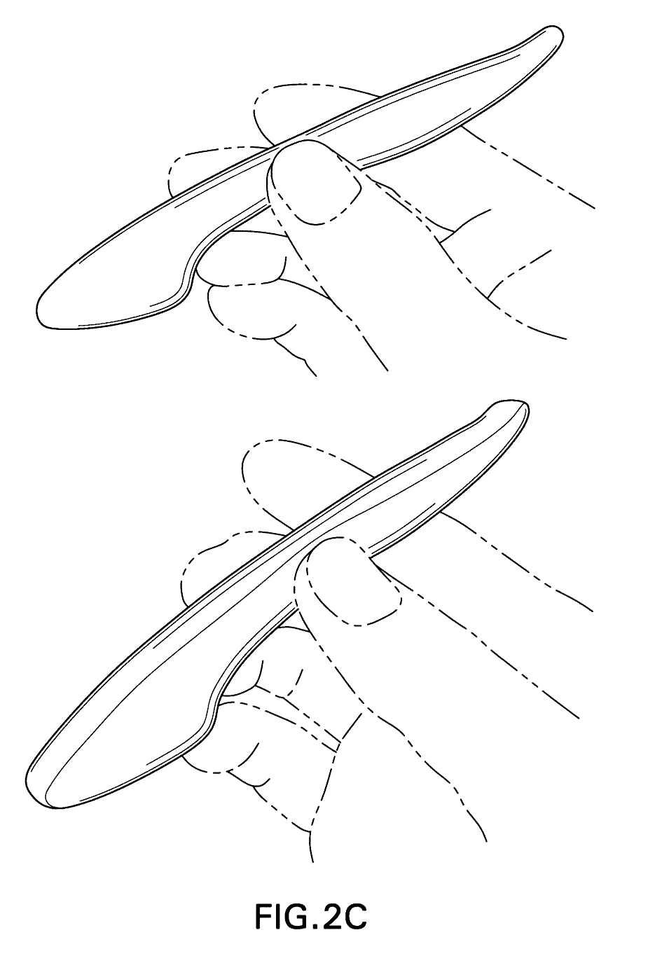

FIGS. 2a-c show illustrative embodiments of the device, potential hand positions and estimated size of the device with respect to the human hand. The figures show a head with 3 contact areas, the neck and handle of the device.

FIG. 2a-c also illustrates how in one embodiment, the device may be a contained within a continuous material that firmly seals the device from outside materials. It has been contemplated that several types of materials could be used to house the device. The materials could include various metals and polyurethane plastics. It has been contemplated that the head of the device could be a metal that is surrounded by a plastic gasket that connects to the neck and body of the device. More complex materials combinations have been contemplated including the potential use of multiple layers of materials. In one embodiment, there could be several layers of materials, including an outer elastic layer surround a compressive layer of a suitable softness/rigidity. On other surfaces the outer layer material will be chosen to reduce dry friction or more efficiently transmit the various energy modalities.

The outer layer of the device may have various designs and colors that could be selected by individual users.

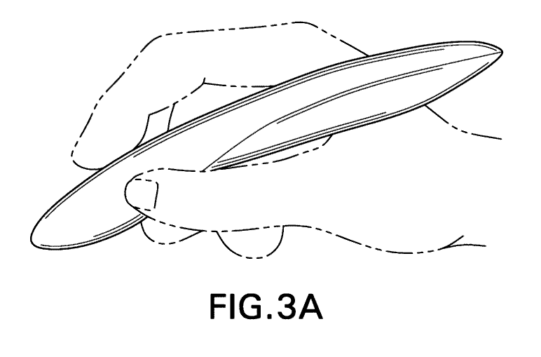

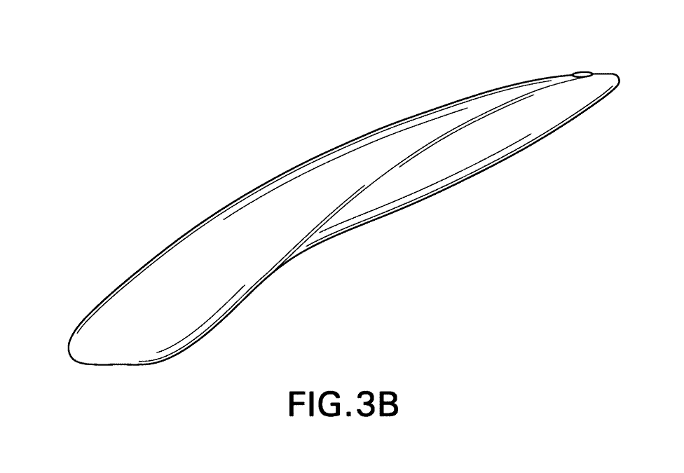

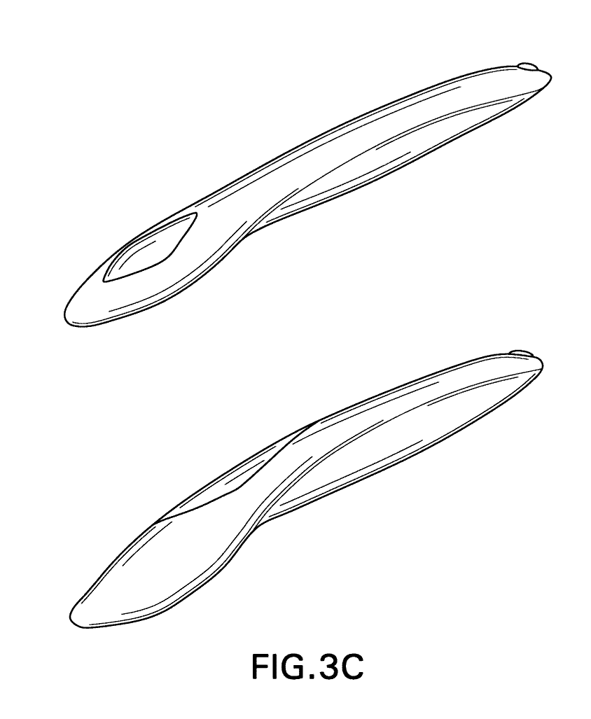

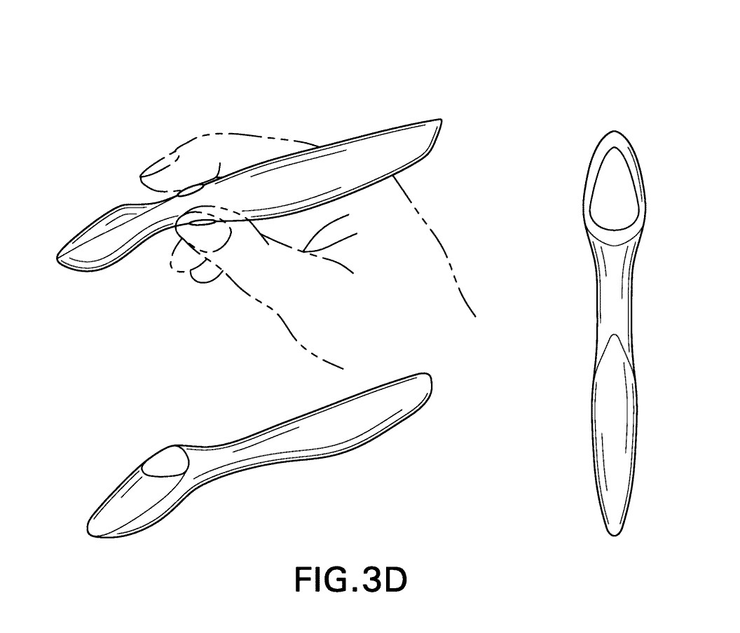

FIGS. 3a-d illustrates alternate embodiments of the device. It has been contemplated that multiple devices could be made available to the user for various types of procedures. It has been contemplated that these devices may function alone or in combination with various lotions and serums that could help to improve the use of the device or better conduct energy from the device to the surface of the skin. These solutions may include various types of conducting and non-conducting gels.

What is claimed is:

1. A portable lightweight handheld integumentary treatment device comprising: a head, a neck and a handle that is intended to be used for contouring, sculpting and finessing cosmetic injectable materials to enhance aesthetic outcomes and is effective in prevention and treatment of lumpiness that may be caused by procedures used to inject cosmetic materials.

2. The device of claim 1 wherein the design of the device and materials encasing the device substantially mirrors the hardness and deformation characteristics of the human thumb or index finger.

3. The device of claim 2 wherein the materials could be multiple layers including an outer and inner layer providing suitable elasticity softness and firmness.

4. The device of claim 1 wherein the head includes a vibrating motor that is operated electrically.

5. The device of claim 1 wherein the head includes various energy modalities or combination of modalities to optimize treatment outcomes including but not limited to: vibration, heating, electrical stimulation, optical illumination, infra-red and ultrasonic energy.

6. The device of claim 2 wherein the materials could be multiple layers including layers providing suitable transmission of various energy modalities noted in claim 5.

7. The device of claim 1 where the head of the device is designed to have several skin contact surfaces including a shorter more acute surface (akin to the palmar surface of the distal thumb), a longer surface (akin to the full length of the index finger) and a curved tip (akin to the tip of either digit) that connects the two faces.

8. The device of claim 1 wherein the neck includes structural and materials design elements to limit the maximal force being transmitted from the handle through the head of the device.

9. The device of claim 1 where the design elements includes a pressure sensor and user signaling modalities including sound, vibration and/or colorimetric signals.

10. The device of claim 1 that can be used in combination with various non-conducting and conducting gels, lotions and serums.
